(19) United States
(12) Patent Application Publication
(10) Pub. No.: US 2015/0096557 A1
(43) Pub. Date: Apr. 9, 2015

(54) LARYNGECTOMEE SNORKEL

(71) Applicant: The Robert H. Bauer Revocable Trust, Hayward, CA (US)

(72) Inventor: Robert H. Bauer, Hayward, CA (US)

(21) Appl. No.: 14/580,203

(22) Filed: Dec. 22, 2014

Related U.S. Application Data
(60) Provisional application No. 61/936,021, filed on Feb. 5, 2014.

(51) Int. Cl. B63C 11/16; A61M 16/04

(52) U.S. Cl. CPC B63C 11/16; A61M 16/0465; B63C 2011/128
USPC 128/201.11

(57) ABSTRACT

Disclosed is a Laryngectomee Snorkel (100) for allowing a Laryngectomee to breathe while swimming. The Laryngectomee Snorkel (100) comprises a Baseplate (110) is configured to have a water-tight seal with a user while allowing a bidirectional passage of air through the Baseplate (110), a Connector (105) configured to have a water-tight seal with the Baseplate (110) and to allow a bidirectional passage of air with the Baseplate (110), an Elbow Tubing (125) configured to have a water-tight seal with the Connector (105) to have a bidirectional passage of air with the Connector (105), a Flexible Riser Tubing (130) configured to have a water-tight seal with the Elbow Tubing (125) and to have a bidirectional passage of air with the Elbow Tubing (125), and an Air Tube (140) configured to have a water-tight seal with the Flexible Riser Tubing (130) and a bidirectional passage of air with the Flexible Riser Tubing (130) and to allow a water-tight bidirectional passage of air with an open atmosphere.

### CROSS-REFERENCE TO RELATED APPLICATION

This application claims the benefit of priority of U.S. Provisional Patent Application 61/936021 filed Feb. 5, 2014 and titled “Laryngectomee Snorkel,” the disclosure of which is incorporated by reference.

### STATEMENT REGARDING FEDERALLY SPONSORED RESEARCH OR DEVELOPMENT

Not Applicable

### BACKGROUND OF THE INVENTION

1. Field of Invention

The invention relates generally to the field of assistive devices and more specifically to devices for Laryngectomees.

2. Description of Related Art

Swimming with a tracheostoma (a hole in the trachea for breathing) requires special caution. The tracheostoma must be occluded to prevent water from entering the lungs directly, but the pathway kept open for breathing. Mouth-connecting snorkels lack the proper fitting and angle to connect with the tracheostoma, while current tracheostoma swimming devices are difficult to use and may be dangerous.

### SUMMARY OF THE INVENTION

Embodiments are directed to a Laryngectomee Snorkel (100) for allowing a Laryngectomee to occlude a stoma while swimming.

The Laryngectomee Snorkel (100) comprises a Baseplate (110) having a Baseplate Neck (150) configured to allow a bidirectional passage of air through the Baseplate (110), with a Baseplate Outer Surface (160) and a Baseplate Skin-Side Surface (165) at a first end of the Baseplate Neck (150) which are configured to securely attach with an air and water-tight seal around a tracheostoma with the Baseplate Neck (150) having a Baseplate Portal (170) proximally located at a center of the first end and configured to allow the bidirectional passage of air to the tracheostoma with a Baseplate Coupling Ring (145) within the Baseplate Neck (150) and proximate to the first end of the Baseplate Neck (150) with the Baseplate Coupling Ring (145) configured for securing the Baseplate (110) to an adapter.

The Laryngectomee Snorkel (100) also comprises a Connector (105) having a Connector Body (105a) and configured as an adapter to the Baseplate (110) and to allow a bidirectional passage of air with the Baseplate (110) with a first end of the Connector Body (105a) having a Baseplate Connector Clip (195) configured to securely attach with a water-tight seal to the Baseplate Coupling Ring (145) with the Connector Body (105a) having a Connector Body Flange (196) medially proximate along the Connector Body (105a) with a Connector Tubing Friction Adapter (197) at a second end configured to create a water-tight seal with a tubing capable of allowing a bidirectional passage of air to and from the Connector (105).

The Laryngectomee Snorkel (100) may also comprise a Tubing Seal (115) configured to seal around the Connector Body (105a) between the Connector Body Flange (196) and the Baseplate (110) for assuring a water-tight seal between the Connector (105) and the Baseplate (110).

The Laryngectomee Snorkel (100) also comprises an Elbow Tubing (125) configured to have a bidirectional passage of air with the Connector (105) and having a first end configured to have a water-tight seal with the Connector (105).

The Laryngectomee Snorkel (100) also comprises a Flexible Riser Tubing (130) configured to have a bidirectional passage of air with the Elbow Tubing (125) and having a first end configured to have a water-tight seal with a second end of the Elbow Tubing (125).

The Laryngectomee Snorkel (100) also comprises an Air Tube (140) configured to have a bidirectional passage of air with the Flexible Riser Tubing (130) and having a first end configured to have a water-tight seal with the Flexible Riser Tubing (130) and having a second end configured to allow a bidirectional passage of air with an open atmosphere.

Various embodiments are disclosed.

### BRIEF DESCRIPTION OF THE DRAWINGS

FIG. 1 shows a view facing a user of an exemplary Laryngectomee Snorkel (100) in place with the user.

FIG. 2 shows the exemplary Laryngectomee Snorkel (100) of FIG. 1 without the user.

FIG. 3 shows a view of the exemplary Laryngectomee Snorkel (100) as might be viewed from a user's tracheostoma with portions cut-away and resized for formatting purposes.

FIGS. 4a and 4b show a side view of an exemplary Laryngectomee Snorkel (100) with

FIG. 4a being an exploded view and FIG. 4b being an assembled view.

FIGS. 5a and 5b shows an embodiment of a Baseplate (110) of an exemplary Laryngectomee Snorkel (100).

FIGS. 6a and 6b show an embodiment of a Neck Strap (120) of an exemplary Laryngectomee Snorkel (100).

FIG. 7 shows an embodiment of a Connector (105) of an exemplary Laryngectomee Snorkel (100).

### DETAILED DESCRIPTION OF THE INVENTION

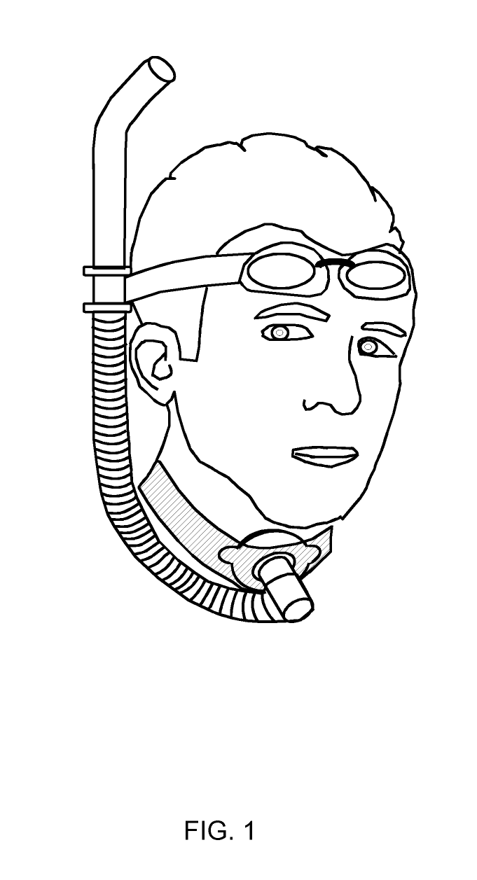

FIG. 1 shows a view facing a user of an exemplary Laryngectomee Snorkel (100) in place with the user. The structures and functions of the exemplary Laryngectomee Snorkel (100) are subsequently detailed.

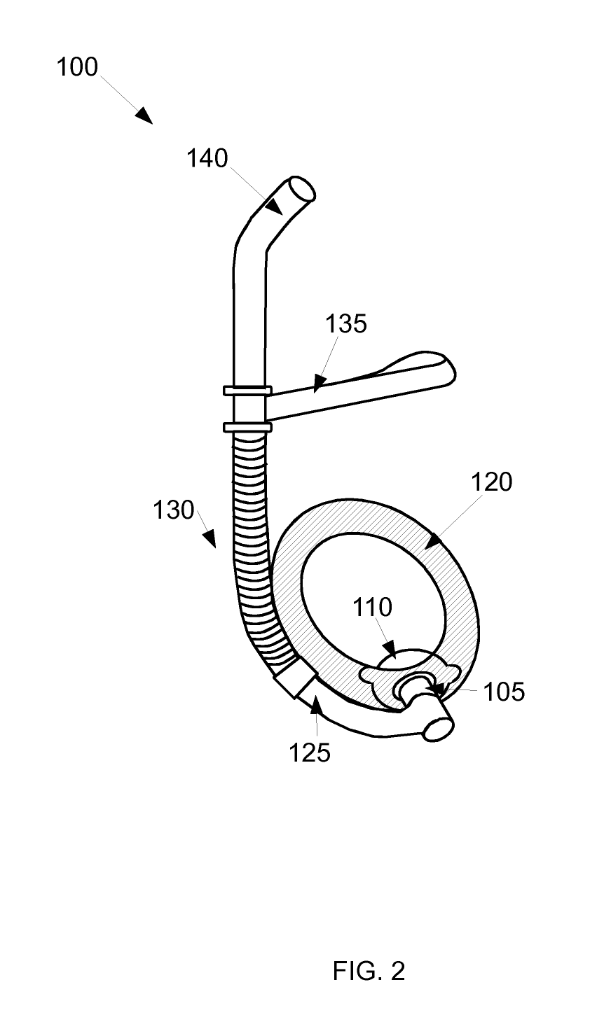

FIG. 2 shows the exemplary Laryngectomee Snorkel (100) of FIG. 1 without the user.

Shown in FIG. 2 and subsequently described in more detail are a Connector (105), a Baseplate (110), a Neck Strap (120), an Elbow Tubing (125), a Flexible Riser Tubing (130), a Mask Strap (135), and an Air Tube (140).

The Connector (105) functions as an adapter between the Baseplate (110) and the other structures of the Laryngectomee Snorkel (100) and provides for a water-tight bidirectional passage of air from above the water to and from the user through the other structures of the Laryngectomee Snorkel (100). The Connector (105) connects to the user via the Baseplate (110) which is securely placed around a laryngectomy stoma of the user.

In some embodiments, the Connector (105) may be commercially acquired, including the Provox® BasePlate Adaptor from Atos Medical AB (Ref. No. 7263). In some embodiments, the Connector (105) may have an elongated configuration to provide for side attachment of the rising portion of the Laryngectomee Snorkel (100). In some embodiments, the Connector (105) may provide for front facing attachment of the rising portion of the Laryngectomee Snorkel (100).

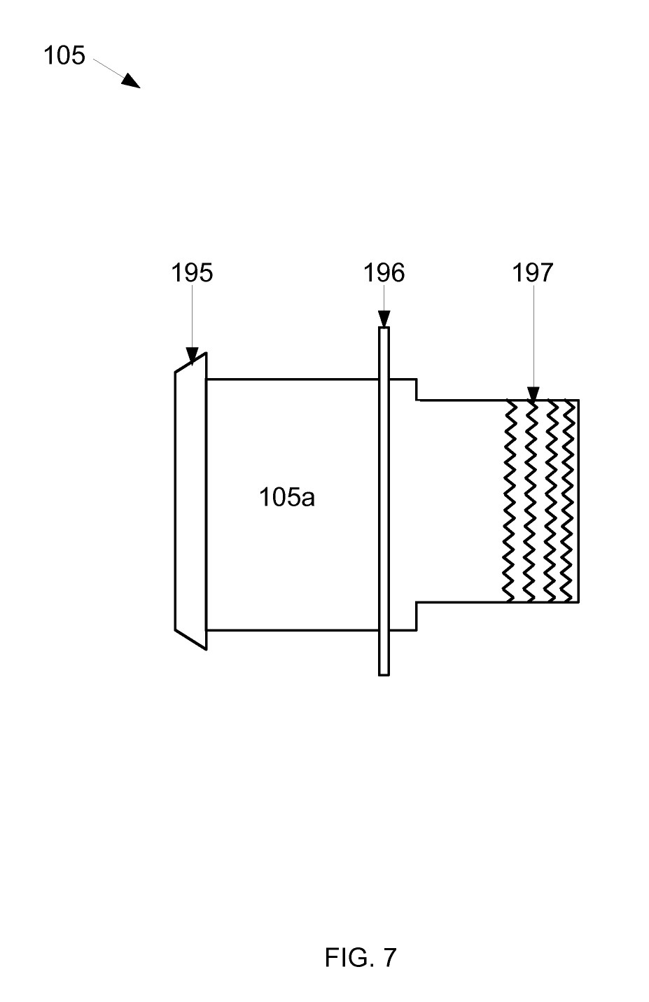

In some embodiments, the Connector (105) could be constructed, provided acceptable requirements are met for sealing integrity with the other structures, non-toxicity and cleanliness for tracheostoma use. The Connector (105) may be from any material capable of providing proper sealing integrity and cleanliness. A firm material is preferable as a flexible plastic would likely distort under pressure. FIG. 7 shows an embodiment of a Connector (105).

The Baseplate (110) provides for a water-tight bidirectional passage of air between the user and the Connector (105). In some embodiments, the Baseplate (110) is commercially available.

Table 1 Commercially Available Baseplates, branded as Provox® StomVent® from Atos Medical AB (Ref. No. 7258):
Flexiderm (oval/round)
Optiderm (oval/round)
Regular (oval/round)
Xtrabase
Stabilibase

Table 2 Commercially Available Baseplates, branded as Blom-Singer® from In-Health

Technologies:
TruSeal (standard)
TruSeal Contour (standard/oval/round)
TruSeal Contour
Low Profile (standard/oval/round)

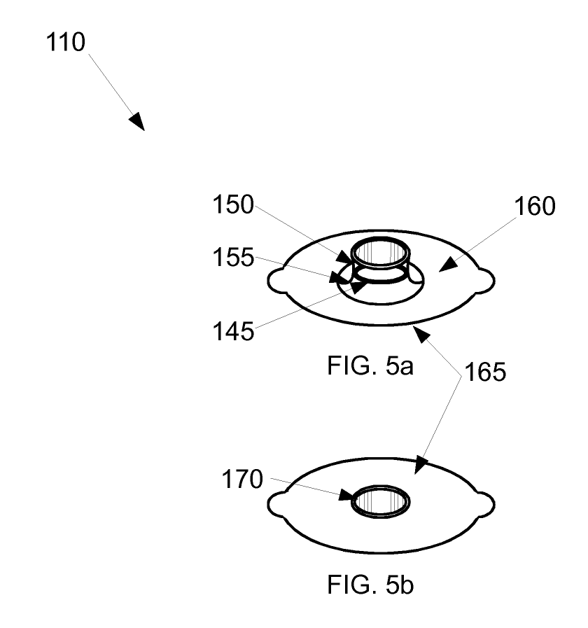

If not commercially acquired, the Baseplate (110) could be constructed separately, provided acceptable standards are met for sealing integrity with the other structures, non-toxicity, non-irritability of the skin and cleanliness for tracheostoma use. FIGS. 5a and 5b show structures of an embodiment of a Baseplate (110).

The Baseplate (110) also has other requirements, which are often cited for user comfort, including a soft fabric material for a lightweight design to contour to the body, a large surface area for better adhesion and helping to prevent air leakage and water incursion, a skin friendly material to reduce skin irritation, a durable material to reduce the need for replacement, and, often, a round or oval shapes for user comfort preference, and sized to the user's stoma.

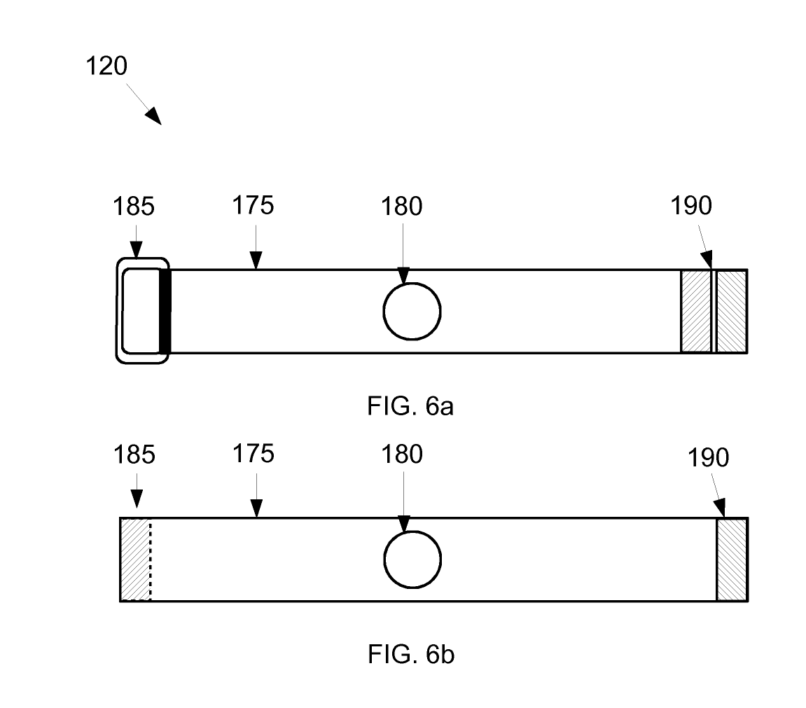

The Neck Strap (120) secures the Laryngectomee Snorkel (100) to the user. The Neck Strap (120) must be comfortable, so it must be flexible and adaptable to variable lengths, preferably in small fractions of an inch. Structure and function of the Neck Strap (120) are subsequently detailed. FIGS. 6a and 6b show an embodiment of a Neck Strap (120) of an exemplary Laryngectomee Snorkel (100) as described below.

The Elbow Tubing (125) provides for a bidirectional passage of air and directs airflow at a first end to and from the Connector (105) at the user's tracheostoma and at a second end to and from the Flexible Riser Tubing (130). As the Elbow Tubing (125) will be underwater, the Elbow Tubing (125) and its connections to the Flexible Riser Tubing (130) and the Connector (105) must be water-tight. In addition, as a user will likely be moving through water, the Elbow Tubing (125) should be able to accommodate user movement and maintain water tightness. Users will typically also desire comfortable use from the Elbow Tubing (125), as by flexing as the user moves. For these reasons, the preferred embodiment of the Elbow Tubing (125) provides some flexibility, as by having a soft plastic construction. Some embodiments of the Elbow Tubing (125) may have hose barbs to secure the Flexible Riser Tubing (130) to the Elbow Tubing (125), while the Connector (105) may have hose barbs to secure to the Elbow Tubing (125).

The Flexible Riser Tubing (130) provides for a bidirectional passage of air at a first end to and from the Elbow Tubing (125), and at a second end to and from the Air Tube (140). The Flexible Riser Tubing (130) also provides flexibility so that the Laryngectomee Snorkel (100) will have the necessary length and position to stay above the water-line. As the Flexible Riser Tubing (130) will be underwater, the Flexible Riser Tubing (130) and its connections must be water-tight. In some embodiments, the Flexible Riser Tubing (130) may be between 15 to 18 inches.

In some embodiments, the Elbow Tubing (125) and the Flexible Riser Tubing (130) are an integrated unit.

The Mask Strap (135) is affixed to the Air Tube (140) for securing the Laryngectomee Snorkel (100) to a head strap, such as is part of swim goggles or a swim mask. In most embodiments, the Mask Strap (135) is at least semi-flexible to permit the Mask Strap (135) to be secured. In some embodiments, the Mask Strap (135) may be a buckle, a hook and loop fastener, e.g., Velcro®, or a loop-pin fastener. The Mask Strap (135) may be two pieces, as with one piece being two inter-connected loops placed around the Air Tube (140), with a second piece looped around the inter-connected piece of the first piece and connected to a mask (not shown).

The Air Tube (140) provides for a bidirectional passage of air and connects at a first end to the Flexible Riser Tubing (130) and rises at a second end above water to allow air to enter into and to exhaust air from the Laryngectomee Snorkel (100). In most embodiments, the Air Tube (140) is at least semi-rigid to maintain an above-the water line aspect with the water. The Air Tube (140) may be semi-flexible for comfortable use, and could have features to permit the Air Tube (140) to be temporarily bent at a specific angle for a specific use. The Air Tube (140) may have a buoyancy attachment to assist in an upright orientation. The Air Tube (140) may have a check-valve to prevent water incursion into the Laryngectomee Snorkel (100).

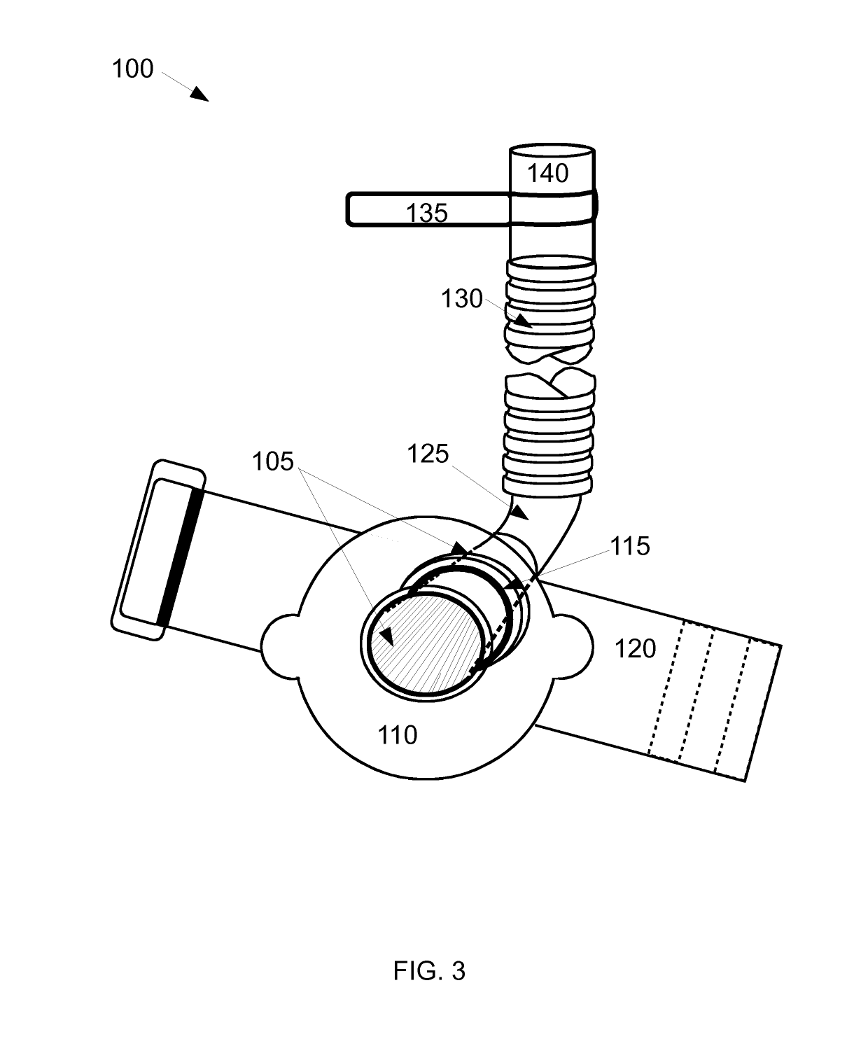

FIG. 3 shows a view of the exemplary Laryngectomee Snorkel (100) as might be viewed from a user's tracheostoma with portions cut-away and resized for viewing purposes. Shown in FIG. 3 are the Connector (105), the Baseplate (110), the Tubing Seal (115), the Neck Strap (120), the Elbow Tubing (125), the Flexible Riser Tubing (130), the Mask Strap (135), and the Air Tube (140).

As shown in FIG. 3, the Connector (105) fits within the Baseplate (110), the structures and functions of which are subsequently detailed. The Neck Strap (120) is behind the Baseplate (110) in this view with one embodiment of a fastening mechanism.

Some embodiments may include a Tubing Seal (115) configured to seal around the Connector Body (105a) between the Connector Body Flange (196) and the Baseplate (110) for assuring sealing integrity (i.e., water-tight) between the Connector (105) and the Baseplate (110). The Tubing Seal could be constructed from any material capable of providing hollow sealing surface around the Connector (105). In some embodiments, the Tubing Seal (115) could be an O-ring. In some embodiments, the O-ring might measure 15/16″ OD× 13/16″ ID× 1/16″. In some embodiments, the Tubing Seal (115) may be a #32 Nitrile Butadiene Rubber O-ring, No. 35746B from Danco Corp.

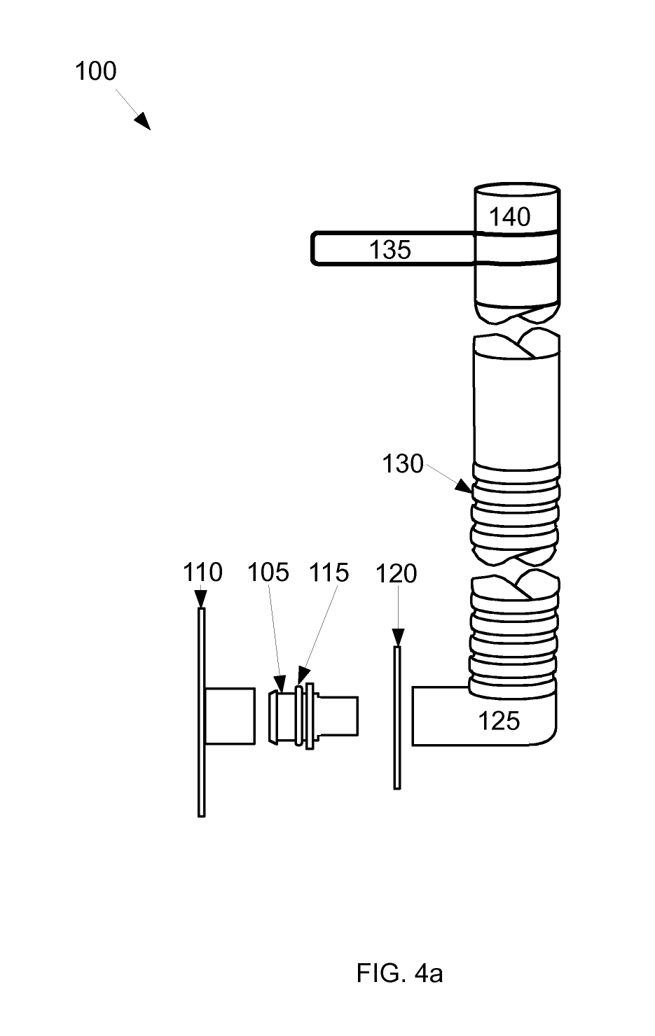

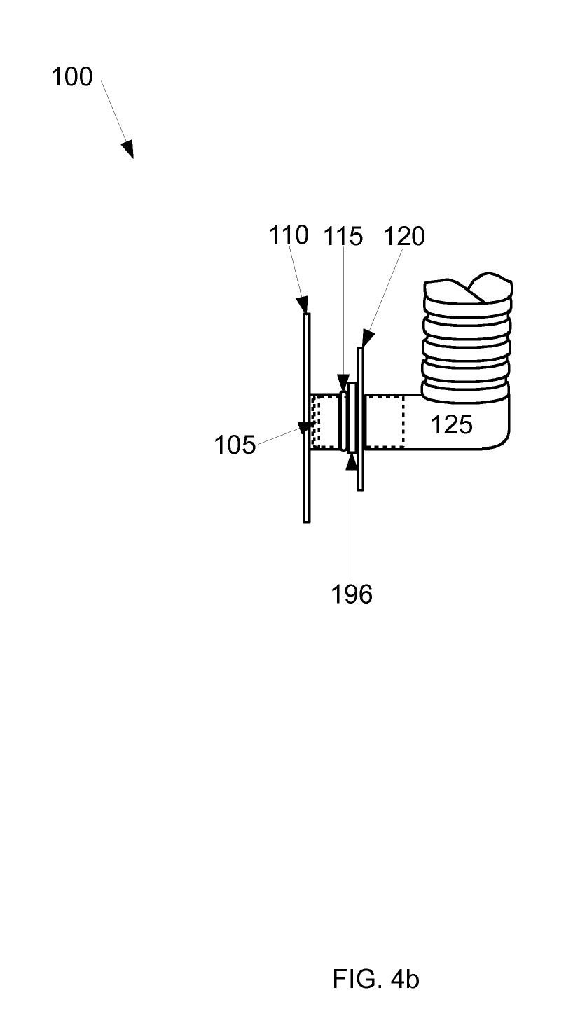

FIGS. 4a and 4b show a side view of an exemplary Laryngectomee Snorkel (100) with FIG. 4a being an exploded view and FIG. 4b being an assembled view. Shown in FIGS. 4a and 4b are the Connector (105), the Baseplate (110), the Tubing Seal (115), the Neck Strap (120), the Elbow Tubing (125), the Flexible Riser Tubing (130), the Mask Strap (135), and the Air Tube (140).

As shown in FIG. 4a, the Connector (105) has a first end connected to the Baseplate (110) and a second end for connecting to the tubing portions of the Laryngectomee Snorkel (100). In some embodiments such as this, the Connector (105) is commercially available as previously described.

The flat side of the Baseplate (110) (a first end, shown in FIG. 5b) would be in place aligned around a user's tracheostoma, with the Baseplate Neck (150) terminating in a second end (shown in FIG. 5a) aligned to receive the Connector (105), (shown in FIG. 7). This positioning of the Baseplate (110) is necessary for the Laryngectomee Snorkel (100) to have an air-tight fit with the user.

The Tubing Seal (115) slips over a first end of the Connector (105) to seal the Connector (105) against the Baseplate (110). Specifically the Tubing Seal (115) is between the Baseplate Neck (150) and a Connector Body Flange (196), shown in FIG. 7. The Neck Strap (120) may then be slipped over a second end of the Connector (105) and placed against the Connector Body Flange (196). A first end of the Elbow Tubing (125) then slips over a second end of the Connector (105) to provide a water-tight seal of the Laryngectomee Snorkel (100) for the user. The Flexible Riser Tubing (130), if not integrated with the Elbow Tubing (125), is then slipped onto to the Elbow Tubing (125).

FIGS. 5a and 5b shows an embodiment of a Baseplate (110) of an exemplary Laryngectomee Snorkel (100).

Shown in FIGS. 5a and 5b are a Baseplate Coupling Ring (145), a Baseplate Neck (150), a Baseplate Sealing Ring (155), a Baseplate Outer Surface (160), a Baseplate Skin-Side Surface (165), and a Baseplate Portal (170).

As shown in FIG. 5a, the Baseplate Coupling Ring (145) is within the Baseplate Neck (150) proximate to the Baseplate Portal (170) and has an annual shape to function as a securing point for the Connector (105) to the Baseplate (110). The Baseplate Coupling Ring (145) must be made from a firm material that will both stay firm for a secure seal, and yet have sufficient yield to engage the Connector (105).

The Baseplate Neck (150) provides spacing for the Baseplate Coupling Ring (145) to engage the Connector (105) (at the Baseplate Connector Clip (195), see FIG. 7) as well as area for a user to hold the Baseplate (110), while, for example, securing the Connector (105) to the Baseplate (110) and the Neck Strap (120) to the Connector (105), and the Laryngectomee Snorkel (100) to the user. The Baseplate Neck (150) must be made from a firm material that will stay firm for a secure seal.

The Baseplate Sealing Ring (155) connects and secures the Baseplate Neck (150) to the Baseplate Skin-Side Surface (165) and the Baseplate Outer Surface (160). The Baseplate Sealing Ring (155) acts as a transition connection between the Baseplate Neck (150) and the Baseplate Skin-Side Surface (165) and the Baseplate Outer Surface (160) to make the connection durable during use.

The Baseplate Outer Surface (160) provides a barrier against deleterious environment conditions, including water, from passing through the Baseplate into a user's tracheostoma. The Baseplate Outer Surface (160) may comprise a flexible plastic for a water-tight seal.

Turning now to FIG. 5b, the Baseplate Skin-Side Surface (165) provides a securing surface for the Baseplate (110) to the skin of a user. The Baseplate Skin-Side Surface (165) typically has a skin-safe adhesive.

The Baseplate Portal (170) provides a secure and non-leaking portal-connection around the tracheostoma and acts as a passageway for air to transport between the user and the atmosphere. In some embodiments, the Baseplate Portal (170) is round with a diameter close to the size of the user's tracheostoma to provide a maximum cross-sectional area and fit with minimal perimeter.

FIGS. 6a and 6b show an embodiment of a Neck Strap (120) of an exemplary Laryngectomee Snorkel (100). Shown in FIGS. 6a and 6b are a Strap Body (175), a Strap Penetration Port (180), a Strap Coupling First End (185), and a Strap Coupling Second End (190).

The Strap Body (175) provides pressure against the user to hold the Laryngectomee Snorkel (100) in place. The Strap Body (175) must be flexible to wrap around a user's neck and hold the Laryngectomee Snorkel (100) in place. In some embodiments, the Strap Body (175) is cloth. In some embodiments, the Strap Body (175) may be another material, including plastic and similar elastomers, or rubber or other elastic materials.

In some embodiments, the Strap Body (175) may be narrow, i.e., approximately in width as the diameter of the Baseplate Portal (170). A Strap Body (175) which is narrow may require higher pressure against the skin to maintain a tight seal of the Connector (105) against a tracheostoma. In some embodiments, the Strap Body (175) may be wide, i.e., approximately twice in width as the diameter of the Baseplate Portal (170). A Strap Body (175) which is wide may require a lower pressure against the skin and yet maintain a tight seal of the Connector (105) against a tracheostoma.

The Strap Penetration Port (180) provides an area for insertion of the Connector (105) through the Neck Strap (120). The Strap Penetration Port (180) should have the same cross-section, i.e., round, which is typical, to provide an optimum securing area for the Neck Strap (120).

The Strap Coupling First End (185) and the Strap Coupling Second End (190) function to secure the Neck Strap (120) in place. Typically the Strap Coupling First End (185) secures to the Strap Coupling Second End (190).

FIG. 6a show an embodiment of the Strap Coupling First End (185) as a D-ring, while the Strap Coupling Second End (190) is a hook and loop fasteners, e.g., Velcro®, which attaches around the D-ring to secure the two-halves of the hook and loop fasteners at the Strap Coupling Second End (190) to the Strap Coupling First End (185). The ends could be reversed.

FIG. 6b shows an embodiment of the Strap Coupling First End (185) as one form of the hook and loop fastener, e.g., the hook, while the Strap Coupling Second End (190) is the other form of the hook and loop fastener, e.g., the loop, so the Strap Coupling First End (185) connects directly to the Strap Coupling Second End (190). In some embodiments, the Strap Coupling First End (185) and the Strap Coupling Second End (190) may use an adhesive to provide for secure use. The ends could be reversed.

FIG. 7 shows an exemplary Connector (105). Shown in FIG. 7 are a Connector Body (105a), a first end - the Baseplate Connector Clip (195), a Connector Body Flange (196) medially proximate along the Connector Body (105a), and a second end—the Connector Tubing Friction Adapter (197).

As stated above, the Connector (105) may be made from any material capable of providing sealing integrity and cleanliness. A firm material is preferable as a flexible material would likely distort under pressure.

The Baseplate Connector Clip (195) provides a secure connection of the Connector (105) and the attached Laryngectomee Snorkel (100) to the Baseplate (110). In some embodiments, the Baseplate Connector Clip (195) has at least one ridge to provide the secure connection.

The Connector (105) also comprises a Connector Tubing Friction Adapter (197) at a second end to provide a secure connection of the Connector (105) to the Elbow Tubing (125) of the Laryngectomee Snorkel (100). In some embodiments, the Connector Tubing Friction Adapter (197) is a friction fit between the Elbow Tubing (125) and the Connector Body (105a). The Connector Tubing Friction Adapter (197) may be smooth. In some embodiments, the Connector Tubing Friction Adapter (197) may be one or more rows of ridges, for example, a hose barb, around a portion of the Connector Body (105a).

These descriptions and drawings are embodiments and teachings of the disclosure. All variations are within the spirit and scope of the disclosure. This disclosure is not to be considered as limiting the claims to only the embodiments illustrated or discussed. Certain changes can be made in the subject matter without departing from the spirit and the scope of this invention. It is realized that changes are possible within the scope of this invention and it is further intended that each structure or element recited in any of the claims is to be understood as referring to all equivalent structure or elements. The following claims are intended to cover the invention as broadly as possible in whatever form it may be used.

What is claimed is:

1. A Laryngectomee Snorkel (100) comprising:
a Baseplate (110) having a Baseplate Neck (150) configured to allow a bidirectional passage of air through the Baseplate (110), with a Baseplate Outer Surface (160) and a Baseplate Skin-Side Surface (165) at a first end of the Baseplate Neck (150) which are configured to securely attach with a water-tight seal around a tracheostoma with the Baseplate Neck (150) having a Baseplate Portal (170) proximally located at a center of the first end and configured to allow the bidirectional passage of air to the tracheostoma with a Baseplate Coupling Ring (145) within the Baseplate Neck (150) and proximate to the first end of the Baseplate Neck (150) with the Baseplate Coupling Ring (145) configured for securing the Baseplate (110) to an adapter,
a Connector (105) having a Connector Body (105a) and configured as an adapter to the Baseplate (110) and to allow a bidirectional passage of air with the Baseplate (110) with a first end of the Connector Body (105a) having a Baseplate Connector Clip (195) configured to securely attach with a water-tight seal to the Baseplate Coupling Ring (145) with the Connector Body (105a) having a Connector Body Flange (196) medially proximate along the Connector Body (105a) with a Connector Tubing Friction Adapter (197) at a second end configured to create a water-tight seal with a tubing configured to have a bidirectional passage of air with the Connector (105),
an Elbow Tubing (125) configured to have a bidirectional passage of air with the Connector (105) and having a first end configured to have a water-tight seal with the Connector (105),
a Flexible Riser Tubing (130) configured to have a bidirectional passage of air with the Elbow Tubing (125) and having a first end configured to have a water-tight seal with a second end of the Elbow Tubing (125), and
an Air Tube (140) configured to have a bidirectional passage of air with the Flexible Riser Tubing (130) and having a first end configured to have a water-tight seal with the Flexible Riser Tubing (130) and having a second end configured to allow a bidirectional passage of air with an open atmosphere.

2. The Laryngectomee Snorkel (100) of claim 1 further comprising a Tubing Seal (115) configured to seal around the Connector Body (105a) between the Connector Body Flange (196) and the Baseplate (110) for assuring a water-tight seal between the Connector (105) and the Baseplate (110).

3. The Laryngectomee Snorkel (100) of claim 1 further comprising a Mask Strap (135) configured to secure the Laryngectomee Snorkel (100) to a head strap.

4. The Laryngectomee Snorkel (100) of claim 1 wherein the Elbow Tubing (125) and the Flexible Riser Tubing (130) are an integrated unit.

5. The Laryngectomee Snorkel (100) of claim 1 wherein the Connector Tubing Friction Adapter (197) comprises a hose barb.

6. The Laryngectomee Snorkel (100) of claim 1 further comprising a Neck Strap (120) capable of securing the Baseplate (110) with the Connector (105) securely attached to the Baseplate (110).

7. The Laryngectomee Snorkel (100) of claim 6 wherein the Neck Strap (120) comprises a Strap Body (175), a Strap Penetration Port (180), a Strap Coupling First End (185) and a Strap Coupling Second End (190).

8. The Neck Strap (120) of claim 7 wherein the Strap Coupling First End (185) and the Strap Coupling Second End (190) comprise a hook and loop fastener.

9. The Neck Strap (120) of claim 7 wherein the Strap Coupling First End (185) comprises a D-ring and the Strap Coupling Second End (190) comprises a hook and loop fastener.

10. The Neck Strap (120) of claim 7 wherein the Strap Coupling First End (185) comprises a hook and loop fastener and the Strap Coupling Second End (190) comprises a D-ring.
